(19) United States
(12) Patent Application Publication
(10) Pub. No.: US 2024/0410607 A1
(43) Pub. Date: Dec. 12, 2024

(54) QUARANTINE AIR-CONDITIONING SYSTEM FOR PREVENTING SPREAD OF AIRBORNE INFECTIOUS DISEASES

(71) Applicant: KOREA INSTITUTE OF MATERIALS SCIENCE, Changwon-si (KR)

(72) Inventors: Seung-hoon LEE, Changwon-si (KR); Do-geun KIM, Changwon-si (KR); Joo-young PARK, Changwon-si (KR); Eun-yeon BYEON, Changwon-si (KR); Sung-hoon JUNG, Changwon-si (KR)

(21) Appl. No.: 18/738,289

(22) Filed: Jun. 10, 2024

(30) Foreign Application Priority Data
Jun. 9, 2023 (KR) 10-2023-0074066

(51) Int. Cl. F24F 11/64; F24F 11/61; F24F 120/10; G16H 50/30

(52) U.S. Cl. CPC F24F 11/64; F24F 11/61; G16H 50/30; F24F 2120/10

(57) ABSTRACT

The present disclosure provides a quarantine air-conditioning system for preventing a spread of airborne infectious diseases, and more particularly, to a quarantine air-conditioning system for preventing a spread of airborne infectious diseases, which may accurately predict a risk of infection with airborne infectious diseases to thus effectively prevent a spread of the airborne infectious diseases, and a method for preventing a spread of airborne infectious diseases using the same.

### CROSS REFERENCE TO RELATED APPLICATION

This application claims the benefit of Korean Patent Application No. 10-2023-0074066, filed on Jun. 9, 2023, which is hereby incorporated by reference in its entirety into this application.

### BACKGROUND

### 1. Field

The present disclosure relates to a quarantine air-conditioning system for preventing a spread of airborne infectious diseases, and more particularly, to a quarantine air-conditioning system for preventing a spread of airborne infectious diseases, which may accurately predict a risk of infection with airborne infectious diseases to thus effectively prevent a spread of the airborne infectious diseases, and a method for preventing a spread of airborne infectious diseases using the same.

### 2. Description of Related Art

Airborne infection may be the most common mode of respiratory infectious diseases. That is, the airborne infection may refer to infection occurring when bacteria or viruses (hereinafter, pathogens) are released in droplets released by a patient while coughing or talking, and then inhaled into a respiratory tract together with air.

SARS coronavirus (SARS COV), which is a representative airborne infectious disease and epidemic in 2002, caused 774 deaths, novel swine-origin influenza A (H1N1), which broke out in 2009, caused more deaths (16,713 deaths, WHO Pandemic (H1N1) 2009-update 91) than the SARS-coronavirus, and the outbreak of Middle East Respiratory Syndrome (MERS), which occurred in 2012 and epidemic in Korea in 2015, resulted in at least 850 deaths. The recent outbreak of coronavirus disease 2019 (COVID-19) has been epidemic for a longer time period than the previous viruses, and caused approximately 6.29 million deaths as of Apr. 5, 2023. Such airborne infectious diseases have been continuously epidemic, it is impossible to rule out a possibility that more diverse types of viruses and mutants will occur in the future. Therefore, there is a need for continued research on a way to countermeasure new types of viruses. In addition, there are limits to 100% prevention of the airborne infection by using masks. Therefore, there is a need for research on a quarantine air-conditioning system for preventing a spread of airborne infectious diseases, which may detect viruses floating in air and immediately respond when the viruses are discovered indoors.

In particular, after the COVID-19 pandemic, expected is an increasing demand for a technology of detecting high-risk viruses floating in indoor air and then predicting spatiotemporal information of virus spread to thus inform an occupant of an infection risk level and quickly purify the indoor air.

However, a current quarantine air-conditioning system may be a combination of a fine dust sensor and a network to a conventional system, and have problems with its purpose of preventing infectious disease spread. In addition, when calculating an airborne virus infection risk, this system may have difficulties in practically and directly reflecting the risk with a spatially averaged result, and may not reflect a feature of each virus type.

In accordance with an increasing social demand for providing an indoor environment safe from the pathogens in addition to the above problems, there is a further increasing need for a quarantine air-conditioning system which may always detect the pathogens to prevent the spread of the airborne infectious diseases.

As a background art of the present disclosure, Korean Patent No. 10-2022-0145459 discloses air conditioning system and method for disinfection of public facilities.

### SUMMARY

An object of the present disclosure is to provide a quarantine air-conditioning system for preventing a spread of airborne infectious diseases, which may accurately predict a risk of infection with airborne infectious diseases to thus effectively prevent a spread of the airborne infectious diseases.

Another object of the present disclosure is to provide a quarantine air-conditioning system for preventing a spread of airborne infectious diseases, which may effectively prevent a spread of airborne infectious diseases by more accurately predicting a risk of infection with the airborne infectious diseases based on a feature of a pathogen, which is a cause of the airborne infectious disease and interacts with an indoor environment.

Still another object of the present disclosure is to provide a quarantine air-conditioning method for preventing a spread of airborne infectious diseases, which may accurately predict a risk of infection with airborne infectious diseases to thus effectively prevent a spread of the airborne infectious diseases.

Other objects and advantages of the present disclosure will be more apparent by the following detailed description, the claims, and the drawings.

According to an aspect, there is provided a quarantine air-conditioning system for preventing a spread of airborne infectious diseases, the system including: an air quality detection unit detecting an air quality of a quarantine air-conditioning target space; a quarantine air-conditioning central control unit issuing a quarantine countermeasure instruction by calculating an airborne infectious disease-infection risk based on air quality information detected by the air quality detection unit; and a quarantine air-conditioner operated based on the instruction issued by the quarantine air-conditioning central control unit, wherein the air quality detection unit includes a complex environmental sensor detecting information on a temperature, humidity, carbon dioxide, a total volatile organic compound (TVOC), particulate matter (PM), or an occupant, and a pathogen sensor detecting airborne infectious pathogens.

According to an embodiment, the pathogen sensor may detect the bacteria, fungi or viruses floating in indoor air, and transmit a detection result to the quarantine air-conditioning central control unit.

According to an embodiment, the one or more air quality detection units may be disposed in the quarantine air-conditioning target space, collect the air quality information at a predetermined time interval, and transmit the collected information to the quarantine air-conditioning central control unit.

According to an embodiment, the quarantine air-conditioning central control unit may include an information processing unit calculating the infection risk for each airborne infectious disease based on the air quality information detected by the complex environmental sensor and the pathogen sensor.

According to an embodiment, the airborne infectious disease-infection risk may be based on an airborne infectious disease-infection probability calculated by Equation 1 below:

$$P = 1 - {e}^{- N} = 1 - {e}^{- IR \times n ( t ) \times t} , \qquad [ Equation 1 ]$$

where P indicates the infection probability (here, 1 indicates 100%), N indicates a total amount of infectious particles (Quanta) inhaled by one person, IR indicates an hourly gas respiration volume (m³/h) of an infected occupant, n(t) indicates Quanta concentration (m⁻³), and t indicates a residence time (h),
the Quanta concentration in Equation 1 is calculated by Equation 2 below:

$$n ( t ) = {n}_{0} {e}^{- \sum λ \times t} + \frac{S}{V \times \sum λ} \times ( 1 - {e}^{- \sum λ \times t} ) \qquad [ Equation 2 ]$$

where n₀ indicates initial Quanta concentration, S indicates Quanta generation amount (1/h), V indicates a volume of an indoor space (m³), and Σλ indicates a total attenuation amount of pathogens per hour, and the total attenuation amount of pathogens per hour in Equation 2 is calculated by Equation 3 below:

$$\sum λ = {λ}_{vent} + {λ}_{RH} + {λ}_{dep} + {λ}_{PAC} + {λ}_{rec} [ \frac{1}{h} ] , \qquad [ Equation 3 ]$$

where λᵥₑₙₜ indicates an amount of infectious particles emitted by ventilation, λ_{RH} indicates an amount of natural pathogen inactivation by relative humidity, λ_{dep} indicates an amount of pathogens falling by gravity, λ_{PAC} indicates an amount of pathogens removed by an indoor quarantine air-conditioner, and λ_{rec} indicates an amount of pathogens removed by a building circulation air conditioner.

According to an embodiment, the airborne infectious disease-infection risk may be based on a total attenuation amount of pathogens calculated by Equation 3 below:

$$\sum λ = {λ}_{vent} + {λ}_{RH} + {λ}_{dep} + {λ}_{PAC} + {λ}_{rec} [ \frac{1}{h} ] , \qquad [ Equation 3 ]$$

where λᵥₑₙₜ indicates an amount of infectious particles emitted by ventilation, λ_{RH} indicates an amount of natural pathogen inactivation by relative humidity, λ_{dep} indicates an amount of pathogens falling by gravity, λ_{PAC} indicates an amount of pathogens removed by an indoor quarantine air-conditioner, and λ_{rec} indicates an amount of pathogens removed by a building circulation air conditioner.

According to an embodiment, the pathogens may be one or more of viruses, bacteria, and fungi.

According to an embodiment, the quarantine air-conditioning central control unit may include a receiver receiving the air quality information detected by the complex environmental sensor and pathogen sensor of the air quality detection unit.

According to an embodiment, the quarantine air-conditioning central control unit may include a controller designed to instruct the quarantine air-conditioner to perform an enhanced quarantine air-conditioning operation when the airborne infectious disease-infection risk is a predetermined reference or higher.

According to an embodiment, the quarantine air-conditioner may receive the instruction from the quarantine air-conditioning central control unit to execute one or more of capture of the infectious particles, inactivation of the captured particles, dehumidification, and a temperature control.

According to an embodiment, the quarantine air-conditioner may be a fixed or mobile type, and include the air quality detection unit.

According to an embodiment, the quarantine air-conditioner may be installed by analyzing the distribution and residence time of the particle spread under a building air-conditioning condition provided by air-conditioning equipment already installed in a building.

According to an embodiment, the quarantine air-conditioning central control unit may display quarantine status notification in real time.

According to another aspect, there is provided a quarantine air-conditioning method using a quarantine air-conditioning system for preventing a spread of airborne infectious diseases of the present disclosure, the method including: i) operating a complex environmental sensor and a pathogen sensor; ii) analyzing, by a quarantine air-conditioning central control unit, air quality information detected by the complex environmental sensor; analyzing, by the quarantine air-conditioning central control unit, a detection result of pathogens detected by the pathogen sensor; iv) calculating, by the quarantine air-conditioning central control unit, an airborne infectious disease-infection risk based on analysis results of steps ii) and iii); v) instructing, by the quarantine air-conditioning central control unit, a quarantine air-conditioner to perform an enhanced operation when the calculated airborne infectious disease-infection risk is a predetermined reference or higher; and vi) notifying, by the quarantine air-conditioning central control unit, the airborne infectious disease-infection risk.

According to an embodiment, the airborne infectious disease-infection risk may be based on an airborne infectious disease-infection probability calculated by Equation 1 below:

$$P = 1 - {e}^{- N} = 1 - {e}^{- IR \times n ( t ) \times t} , \qquad [ Equation 1 ]$$

where P indicates the infection probability (here, indicates 100%), N indicates a total amount of infectious particles (Quanta) inhaled by one person, IR indicates an hourly gas respiration volume (m³/h) of an infected occupant, n(t) indicates Quanta concentration (m⁻³), and t indicates a residence time (h),

the Quanta concentration in Equation 1 is calculated by Equation 2 below:

$$n ( t ) = {n}_{0} {e}^{- \sum λ \times t} + \frac{S}{V \times \sum λ} \times ( 1 - {e}^{- \sum λ \times t} ) , \qquad [ Equation 2 ]$$

where n₀ indicates initial Quanta concentration, S indicates Quanta generation amount (1/h), V indicates a volume of an indoor space (m³), and Σλ indicates a total attenuation amount of pathogens per hour, and

the total attenuation amount of pathogens per hour in Equation 2 is calculated by Equation 3 below:

$$\sum λ = {λ}_{vent} + {λ}_{RH} + {λ}_{dep} + {λ}_{PAC} + {λ}_{rec} [ \frac{1}{h} ] , \qquad [ Equation 3 ]$$

where λᵥₑₙₜ indicates an amount of infectious particles emitted by ventilation, λ_{RH} indicates an amount of natural pathogen inactivation by relative humidity, λ_{dep} indicates an amount of pathogens falling by gravity, λ_{PAC} indicates an amount of pathogens removed by an indoor quarantine air-conditioner, and λ_{rec} indicates an amount of pathogens removed by a building circulation air conditioner.

According to still another aspect, there is provided a system for predicting infection with airborne infectious diseases, the system including an air quality detection unit detecting an air quality of a quarantine air-conditioning target space; and a quarantine air-conditioning central control unit predicting an airborne infectious disease-infection risk based on air quality information detected by the air quality detection unit and past air quality information already input, wherein the air quality detection unit includes a complex environmental sensor detecting information on a temperature, humidity, carbon dioxide, a total volatile organic compound (TVOC), particulate matter (PM), or an occupant, and a pathogen sensor detecting airborne infectious pathogens, and the quarantine air-conditioning central control unit predicts the airborne infectious disease-infection risk by an indoor air quality prediction algorithm based on time series analysis.

According to an embodiment, the indoor air quality prediction algorithm may be based on autoregressive integrated moving average (ARIMA).

### BRIEF DESCRIPTION OF THE DRAWINGS

FIG. 1 is a schematic diagram of a quarantine air-conditioning system for preventing a spread of airborne infectious diseases according to an embodiment of the present disclosure.

FIG. 2 is a flow chart schematically showing a quarantine air-conditioning method for preventing a spread of airborne infectious diseases according to an embodiment of the present disclosure.

FIG. 3A is a graph showing an airborne infectious disease-infection probability based on an exposure time and a flow rate of a mobile quarantine air-conditioner (PAC) according to an embodiment of the present disclosure.

FIG. 3B is a graph showing airborne infectious disease-infection probability based on the exposure time and a ventilation flow rate according to an embodiment of the present disclosure.

FIG. 4 is a graph showing the airborne infectious disease-infection probability based on the flow rate of the mobile quarantine air-conditioner (PAC) and ventilation flow rate according to an embodiment of the present disclosure.

FIG. 5 is a diagram showing indoor airflow and particle spread behavior for optimal installation of the mobile quarantine air-conditioner according to an embodiment of the present disclosure.

FIG. 6 is a graph showing PM2.5 sensor data based on a time for each zone of Gyeongsangnam-do Provincial Government Civil Affairs Office.

FIG. 7 is a graph showing time series decomposition in the PM2.5 sensor data of the Gyeongsangnam-do Provincial Government Civil Affairs Office.

FIG. 8 is a graph confirming time series stationarity (auto correlation function (ACF)) in the PM2.5 sensor data of the Gyeongsangnam-do Provincial Government Civil Affairs Office.

FIG. 9 is a graph showing a mean squared error of an autoregressive integrated moving average (ARIMA) (p,d,q) model according to an embodiment of the present disclosure.

FIG. 10 is a graph showing the airborne infectious disease-infection probability and the airborne infectious disease-future infection probability based on various environments and parameters of a heating, ventilation, & air conditioning (HVAC) technology according to an embodiment of the present disclosure.

### DETAILED DESCRIPTION OF THE DISCLOSURE

Terms used in this application are used only to describe the specific embodiments rather than limiting the present disclosure. A term of a single number may include its plural number unless explicitly indicated otherwise in the context.

It is to be understood that terms such as “include”, “have” and the like used in this application, specify the presence of features, numerals, steps, operations, components, parts, or combinations thereof, mentioned in the specification, and do not preclude the presence or addition of one or more other features, numerals, steps, operations, components, parts or combinations thereof.

Unless explicitly described to the contrary, “including” any components will be understood to imply the inclusion of other components rather than the exclusion of any other components. In addition, throughout the specification, the word “on” does not necessarily indicate that any element is disposed on an upper side of a target portion in a gravity direction, and indicates that any element is disposed on/above or beneath/below the target portion.

In this specification, descriptions such as “for example” and the like may not accurately match the information presented here, such as cited features, variables, or values. Embodiments of the invention according to the various embodiments of the present disclosure should not be limited by effects of variations, including tolerances, measurement errors, limits in measurement accuracy, and other commonly known factors.

The present disclosure may be variously modified and have various embodiments, and specific embodiments are shown in the drawings and described in detail in the detailed description. However, it is to be understood that the present disclosure is not limited to specific embodiments, and includes all modifications, equivalents and substitutions, included in the spirit and scope of the present disclosure. When it is decided that the detailed description of the known art related to the present disclosure may obscure the gist of the present disclosure, the detailed description thereof will be omitted.

In this specification, “airborne infectious diseases” may refer to diseases in which the pathogens survive in air by floating through droplet nuclei, dust, or the like, and then penetrate deep into a human body when a human breathes and are spread.

In this specification, “pathogens” may refer to microorganisms causing diseases such as viruses, bacteria, and fungi. The pathogens may generally include anything which may cause the disease.

In addition, in this specification, a “system” may refer to a collection of related elements combined to each other based on a certain rule to realize a necessary function, and the meaning of the system may also be used interchangeably with the meaning of devices organized and regularly interacting with each other to perform a designated information processing function.

### [Quarantine Air-Conditioning System for Preventing Spread of Airborne Infectious Diseases]

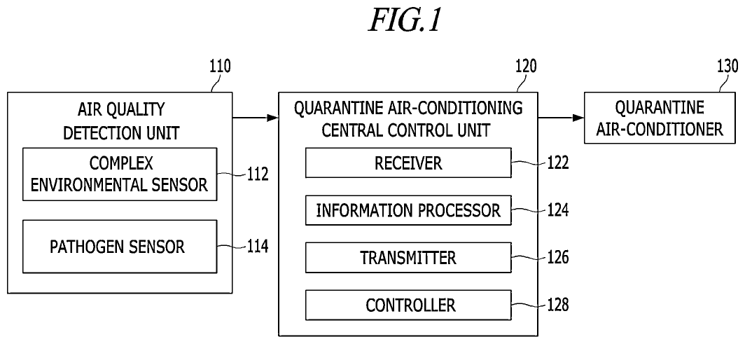

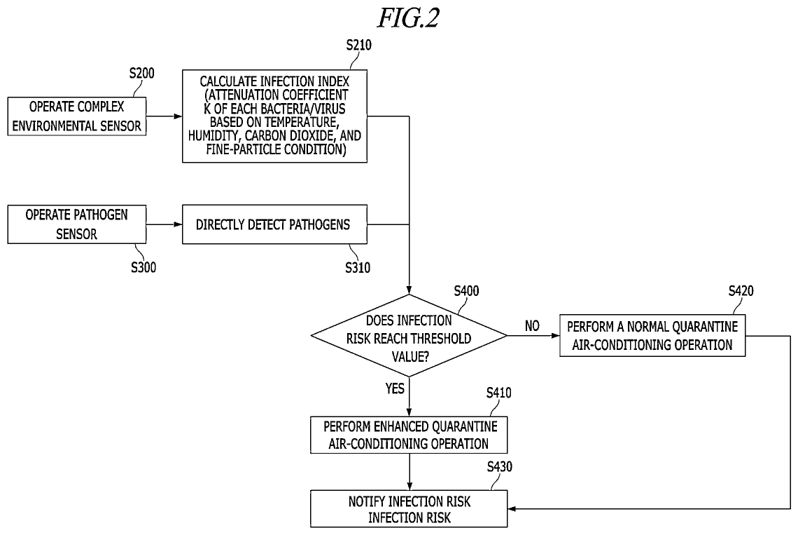

FIG. 1 is a schematic diagram of a quarantine air-conditioning system for preventing a spread of airborne infectious diseases according to an embodiment of the present disclosure. FIG. 2 is a flow chart schematically showing a quarantine air-conditioning method for preventing a spread of airborne infectious diseases according to an embodiment of the present disclosure.

Referring to FIGS. 1 and 2, the quarantine air-conditioning system for preventing a spread of airborne infectious diseases of the present disclosure may include an air quality detection unit 110 detecting an air quality of a quarantine air-conditioning target space; a quarantine air-conditioning central control unit 120 issuing a quarantine countermeasure instruction by calculating an airborne infectious disease-infection risk based on air quality information detected by the air quality detection unit 110, and a quarantine air-conditioner 130 operated based on the instruction issued by the quarantine air-conditioning central control unit 120.

The air quality detection unit 110 may include a complex environmental sensor 112 and a pathogen sensor 114.

The complex environmental sensor 112 of the air quality detection unit 110 may detect information to calculate the airborne infectious disease-infection risk, and transmit the detected information to the quarantine air-conditioning central control unit 120.

The present disclosure is not limited thereto, and the information detected by the complex environmental sensor 112 of the air quality detection unit 110 and then transmitted to the quarantine air-conditioning central control unit 120 may include all information helpful in calculating the airborne infectious disease-infection risk.

The present disclosure is not limited thereto, and the information may include information on a temperature, humidity, carbon dioxide, a total volatile organic compound (TVOC), particulate matter (PM) (or a fine particle), or an occupant.

The present disclosure is not limited thereto, and the fine particle may be at least one of PM10, PM2.5, and a particle having a size of 100 to 1000 nm.

The present disclosure is not limited thereto, and the volatile organic compound may be formaldehyde, toluene, xylene, or a combination thereof.

The present disclosure is not limited thereto, and the occupant information may include the number of occupants, residence time, or the like.

The temperature, humidity, carbon dioxide, total volatile organic compound (TVOC), fine particle (PM), or occupant information may affect the survival and spread of the pathogen which is a cause of the airborne infectious disease, affect the infectious particle (Quanta) inhalation and respiration volume of the occupant, thereby affecting an infection probability.

The temperature and humidity may be used to calculate a natural reduction rate for each pathogen based on the temperature/humidity and time that is stored in an information processing unit 124, and used to determine a λ_{RH} value in Equation 3.

Fine particle concentration information may be used in a Quanta concentration model through a fine particle concentration stored in the information processor 124 to thus derive an S value in Equation 2. The Quanta concentration model for each fine particle concentration may be derived through data from the complex environmental sensor 112 and an empirical formula acquired through experiments performed using an impactor or an impinger, which is a floating pathogen measurement device.

The pathogen sensor 114 of the air quality detection unit 110 may detect the pathogen floating in the indoor air, and transmit a detection result to the quarantine air-conditioning central control unit 120.

The present disclosure is not limited thereto, and the pathogens may be one or more of viruses, bacteria, and fungi.

The pathogen sensor 114 may detect the pathogens using a known pathogen sensor technology and perform qualitative and quantitative analysis of the pathogens. The present disclosure is not limited thereto, and the pathogen sensor 114 may include a lateral flow assay cartridge capable of detecting various types of pathogens and a camera unit capturing a detection result of the pathogens on the cartridge and transmitting the same to the quarantine air-conditioning central control unit 120.

The present disclosure is not limited thereto, and it may be suitable that the pathogen sensor 114 is a virus sensor. According to the above configuration, it is possible to quickly and efficiently quarantine the infectious disease caused by viruses whose resulting damage is enormous due to a rapid progress of its airborne spread.

The present disclosure is not limited thereto, and the pathogen sensor 114 may detect influenza virus, avian influenza virus, SARS-COV-2, respiratory syncytial virus (RSV), or the like.

One or more air quality detection units 110 may be disposed in the quarantine air-conditioning target space, collect the air quality information at a predetermined time interval, and transmit the collected information to the quarantine air-conditioning central control unit 120. According to the above configuration, it is possible to accurately detect the airborne pathogens present in all zones in the quarantine air-conditioning target space in real time, thus more accurately calculating the airborne infectious disease-infection risk.

The present disclosure is not limited thereto, and the air quality detection unit 110 may be installed in a fixed or mobile manner in the quarantine air-conditioning target space. The present disclosure is not limited thereto, and when using the mobile air quality detection unit 110, it is possible to accurately detect the airborne pathogens present in the quarantine air-conditioning target space in real time, thereby accurately calculating the airborne infectious disease-infection risk while minimizing the number of the air quality detection units 110 installed in the quarantine air-conditioning target space.

The present disclosure is not limited thereto, and the quarantine air-conditioning central control unit 120 may include a receiver 122, an information processor 124, a transmitter 126, and a controller 128.

The receiver 122 may receive the air quality information detected by the complex environmental sensor 112 and pathogen sensor 114 of the air quality detection unit 110, and transmit the same to the information processor 124.

The information processor 124 may calculate the infection risk for each airborne infectious disease based on the air quality information detected by the complex environmental sensor 112 and the pathogen sensor 114. The airborne infectious disease-infection risk may be calculated based on the infection probability for each airborne infectious disease.

The present disclosure is not limited thereto, and the information processor 124 may calculate an infection index for each airborne infectious disease or the like based on the temperature, humidity, carbon dioxide, total volatile organic compound (TVOC), fine particle (PM) or occupant information, detected by the complex environmental sensor 112. In more detail, the temperature, humidity, carbon dioxide, or total volatile organic compound (TVOC) information may be used to derive λ_{RH} in Equation 3, which is a natural reduction amount of each pathogen floating in indoor air. Three types of fine particle (PM) concentration may be measured: particles less than 1 μm, PM2.5, and PM10, and may be used to acquire n₀ in Equation 2 from the fine particle (PM) concentration by deriving a correlation between concentration of each particle and floating pathogen concentration measured by the impactor or the impinger. The occupant information may be used to acquire the S value, which refers to an amount of infectious particles emitted by the occupant.

The present disclosure is not limited thereto, and in the quarantine air-conditioning system for preventing a spread of airborne infectious diseases according to the present disclosure, the airborne infectious disease-infection risk may be based on the airborne infectious disease-infection probability calculated by Equation 1 below:

$$P = 1 - {e}^{- N} = 1 - {e}^{- IR \times n ( t ) \times t} . \qquad [ Equation 1 ]$$

In Equation 1 above, P indicates the infection probability (here, 1 indicates 100%), N indicates a total amount of infectious particles (Quanta) inhaled by one person, IR indicates an hourly gas respiration volume (m³/h) of an infected occupant, n(t) indicates Quanta concentration (m⁻³), and t indicates a residence time (h).

As described above, the airborne infectious disease-infection probability may be higher as the total amount of infectious particles inhaled by one person is greater, the hourly gas respiration volume of the infected occupant is greater, the Quanta concentration is higher, and the residence time of the infected occupant is longer.

The Quanta concentration in Equation 1 above may be calculated by Equation 2 below:

$$n ( t ) = {n}_{0} {e}^{- \sum λ \times t} + \frac{S}{V \times \sum λ} \times ( 1 - {e}^{- \sum λ \times t} ) . \qquad [ Equation 2 ]$$

In Equation 2 above, no indicates initial Quanta concentration, S indicates Quanta generation amount (1/h), V indicates a volume of an indoor space (m³), and Σλ indicates a total attenuation amount of pathogens per hour that is calculated by Equation 3 below.

As described above, the Quanta concentration (n(t)) may be decreased as the Quanta generation amount is smaller, the volume of the indoor space is larger, and the total attenuation amount of pathogens per hour is larger.

The present disclosure is not limited thereto, and (n(t)) may be calculated by a correlation equation between the PM concentration and the floating pathogen concentration. A correlation between the PM concentration and the floating pathogen concentration may depend on a location. Therefore, it is necessary to establish an experiment database for each location or type in order to make accurate predictions.

The present disclosure is not limited thereto, and n(t) may be calculated based on the numbers of occupants, mask wearing rate, and filtration rate.

$$\sum λ = {λ}_{vent} + {λ}_{RH} + {λ}_{dep} + {λ}_{PAC} + {λ}_{rec} [ \frac{1}{h} ] . \qquad [ Equation 3 ]$$

In Equation 3 above, λᵥₑₙₜ indicates an amount of infectious particles emitted by ventilation, λ_{RH} indicates an amount of natural pathogen inactivation by relative humidity, λ_{dep} indicates an amount of pathogens falling by gravity, λ_{PAC} indicates an amount of pathogens removed by an indoor quarantine air-conditioner, and λ_{rec} indicates an amount of pathogens removed by a building circulation air conditioner.

As described above, the total attenuation amount of pathogens may be greater as an amount of ventilation is greater, the amount of natural pathogen inactivation by the relative humidity is greater, the amount of pathogens falling by the gravity is greater, the amount of pathogens removed by the indoor quarantine air-conditioner is greater and the air conditioning equipment already installed in a building is greater.

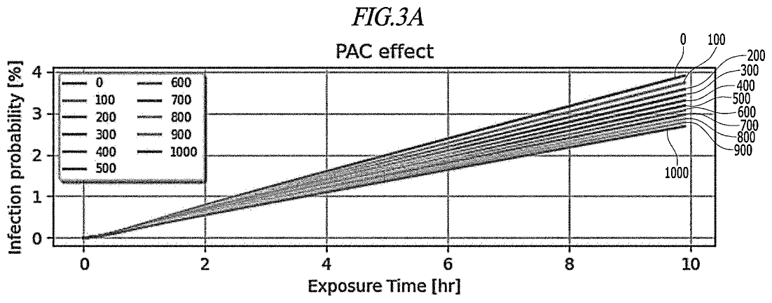

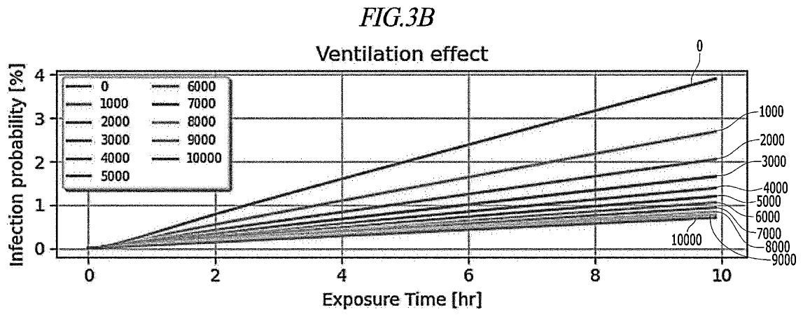

FIG. 3A is a graph showing the airborne infectious disease-infection probability based on an exposure time and a flow rate of the mobile quarantine air-conditioner (PAC) according to an embodiment of the present disclosure, and FIG. 3B is a graph showing the airborne infectious disease-infection probability based on the exposure time and a ventilation flow rate according to an embodiment of the present disclosure. As shown in the graph, it may be seen that the airborne infectious disease-infection probability is decreased as the flow rate of the mobile quarantine air-conditioner or the ventilation flow rate is increased and this decrease becomes more noticeable as the exposure time is longer. The exposure time may be the residence time of the pathogens in the space where the pathogens present.

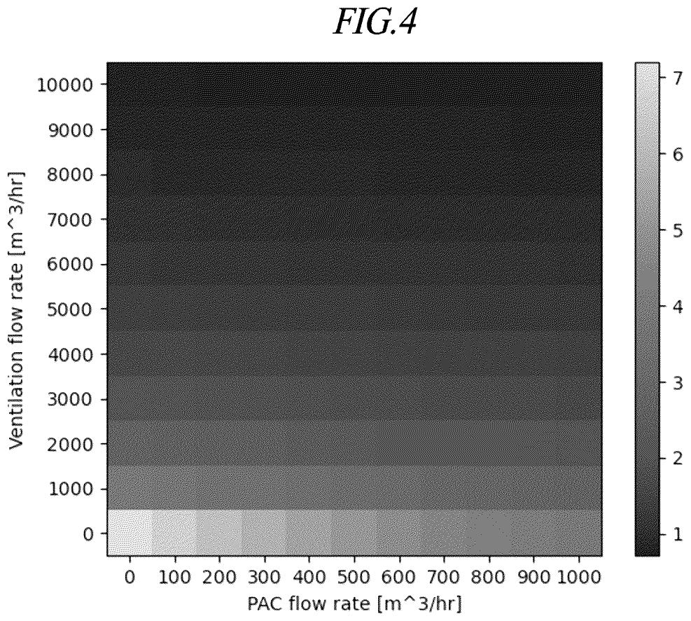

FIG. 4 is a graph showing the airborne infectious disease-infection probability based on the flow rate of the mobile quarantine air-conditioner (PAC) and the ventilation flow rate according to an embodiment of the present disclosure. Referring to FIG. 4, it may be seen that the airborne infectious disease-infection probability is more effectively decreased when simultaneously performing the indoor ventilation than when only operating the mobile quarantine air-conditioner.

The present disclosure is not limited thereto, and the amount of natural pathogen inactivation may be calculated by establishing a half-life database (DB) for each pathogen based on an environmental condition such as the relative humidity or by means of deep learning using artificial intelligence (AI). In addition, when the humidity and temperature of a quarantine target space is changed, it is necessary to reflect a pathogen inactivation value based on the humidity and temperature of the quarantine target space.

According to the above configuration, it is possible to accurately calculate the infectious disease-infection risk caused by the airborne pathogens which may be changed based on various variables in the quarantine target space. The air quality may be abnormal in the space where the actually infected occupant is present. Therefore, it may be unrealistic to calculate the infection index by targeting a stable air quality. However, when using the Equations configured as above, it is possible to calculate the airborne infectious disease-infection risk based on an air quality condition changed in real time depending on building, space, air conditioner, or the like, thus allowing the system of the present disclosure to be used in even an abnormal situation.

The transmitter 126 may issue a detection instruction to the air quality detection unit 110 to further identify a space having a high infection risk when the airborne infectious disease-infection risk is a predetermined reference or higher. According to the above configuration, it is possible to comprehensively identify the various variables that may occur in the quarantine target space, such as movement of the occupant, more accurately calculate the airborne infectious disease-infection risk which may be changed by the above variables.

The controller 128 may be designed to instruct the quarantine air-conditioner 130 to perform an enhanced quarantine air-conditioning operation when the airborne infectious disease-infection risk is the predetermined reference or higher. According to the above configuration, is possible to efficiently operate the quarantine air-conditioner 130, predict a wide spread of the airborne pathogens from a remote location, and provide a site with risk information such as an evacuation instruction, thereby minimizing human damage caused by the wide spread pathogens.

The quarantine air-conditioning central control unit 120 may include a status board for displaying quarantine status notification in real time. According to the above configuration, it is possible to receive the air quality information detected at the site from the remote location, and thus continuously monitor the airborne infectious disease-infection risk in the remote location. Therefore, it is possible to predict the wide spread of the airborne infectious diseases in the remote location, and provide the risk information, such as the evacuation instruction, to the site, thereby minimizing the human damage caused by the wide spread airborne infectious diseases.

The quarantine air-conditioner 130 may receive the instruction from the quarantine air-conditioning central control unit 120 to execute one or more of capture of the infectious particles, inactivation of the captured particles, dehumidification, and a temperature control. According to the above configuration, it is possible to lower the airborne infectious disease-infection risk.

The quarantine air-conditioner 130 may be a fixed or mobile type. The fixed quarantine air-conditioner may include the air conditioning equipment already installed in a building, which is installed in a duct in the building.

When the quarantine air-conditioner 130 is the mobile type, there is no need to install the plurality of quarantine air-conditioners 130, which may reduce its installation cost.

In addition, the quarantine air-conditioner 130 may be the mobile type, and integrated to include the air quality detection unit 110. In this case, the quarantine air-conditioner 130 may be moved to a specific zone, if necessary, to more accurately detect the air quality information, and immediately perform the quarantine when the airborne infectious disease-infection risk in the detected zone is the predetermined reference or higher, thereby further improving quarantine efficiency.

The quarantine air-conditioner 130 may be installed by analyzing the distribution and residence time of the particle spread under a building air-conditioning condition provided by the air-conditioning equipment already installed in a building. According to the above configuration, it is possible to further improve the quarantine efficiency while minimizing the number of the quarantine air-conditioners 130.

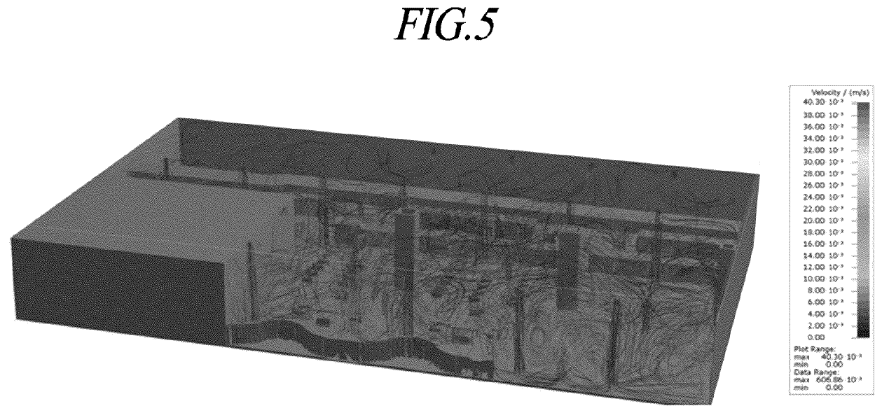

FIG. 5 is a diagram showing indoor airflow and particle spread behavior for optimal installation of the mobile quarantine air-conditioner according to an embodiment of the present disclosure. The present disclosure is not limited thereto, and when utilizing a digital twin manner, it is possible to analyze the distribution and residence time of the particles spread under the building air-conditioning condition to thus derive an effective installation location of the mobile or fixed air conditioner of the air quality detection unit 110.

### [Quarantine Air-Conditioning Method for Preventing Spread of Airborne Infectious Diseases]

Referring to FIGS. 1 and 2, according to another aspect of the present disclosure, provided is a quarantine air-conditioning method using a quarantine air-conditioning system for preventing a spread of airborne infectious diseases, the method including: i) operating a complex environmental sensor and a pathogen sensor; ii) analyzing, by a quarantine air-conditioning central control unit, air quality information detected by the complex environmental sensor; iii) analyzing, by the quarantine air-conditioning central control unit, a detection result of pathogens detected by the pathogen sensor; iv) calculating, by the quarantine air-conditioning central control unit, an airborne infectious disease-infection risk based on analysis results of steps ii) and iii); v) instructing, by the quarantine air-conditioning central control unit, a quarantine air-conditioner to perform an enhanced operation when the calculated airborne infectious disease-infection risk is a predetermined reference or higher; and vi) notifying, by the quarantine air-conditioning central control unit, the airborne infectious disease-infection risk.
Step i) may include operating a complex environmental sensor 112 (S200) and operating a pathogen sensor 114 (S300).
Step ii) indicates step S210 of analyzing, by a quarantine air-conditioning central control unit 120, air quality information based on the air quality information detected by the complex environmental sensor 112.

The present disclosure is not limited thereto, and in this step, it is possible to calculate an infection index for each airborne infectious disease or the like based on information on a temperature, humidity, carbon dioxide, a total volatile organic compound (TVOC), a fine particle (PM), or an occupant, detected by the complex environmental sensor 112.
Step iii) indicates step (S310) of analyzing, by the quarantine air-conditioning central control unit, the detect result of the pathogens detected by the pathogen sensor. The present disclosure is not limited thereto, and in this step, the pathogen sensor may capture the detection result of the pathogens on a lateral flow assay cartridge capable of detecting various types of pathogens and transmit the same to a receiver 122 of the quarantine air-conditioning central control unit 120. In this step, it is possible to perform qualitative and quantitative analysis on the presence and concentration of the pathogens present in a quarantine target space, based thereon. The present disclosure is not limited thereto, and in this step, it is possible to calculate an attenuation coefficient k of each pathogens detected based on the air quality information such as the temperature, humidity, carbon dioxide, total volatile organic compound and fine particles detected and received by the complex environmental sensor 112.
Step iv) indicates step (S400) of calculating, by the information processor 124 of the quarantine air-conditioning central control unit 120, the airborne infectious disease-infection risk based on the analysis results of steps ii) and iii).

The present disclosure is not limited thereto, and in the quarantine air-conditioning method for preventing a spread of airborne infectious diseases, the airborne infectious disease-infection risk may be predicted based on an airborne infectious disease-infection probability calculated by Equation 1 below:

$$P = 1 - {e}^{- N} = 1 - {e}^{- IR \times n ( t ) \times t} . \qquad [ Equation 1 ]$$

In Equation 1 above, P indicates the infection probability (here, 1 indicates 100%), N indicates a total amount of infectious particles (Quanta) inhaled by one person, IR indicates an hourly gas respiration volume (m³/h) of an infected occupant, n(t) indicates Quanta concentration (m⁻³), and t indicates a residence time (h).

The Quanta concentration in Equation 1 above may be calculated by Equation 2 below:

$$n ( t ) = {n}_{0} {e}^{- \sum λ \times t} + \frac{S}{V \times \sum λ} \times ( 1 - {e}^{- \sum λ \times t} ) . \qquad [ Equation 2 ]$$

In Equation 2 above, no indicates initial Quanta concentration, S indicates Quanta generation amount (1/h), V indicates a volume of an indoor space (m³), and Σλ indicates a total attenuation amount of the pathogens per hour that is calculated by Equation 3 below:

$$\sum λ = {λ}_{vent} + {λ}_{RH} + {λ}_{dep} + {λ}_{PAC} + {λ}_{rec} [ \frac{1}{h} ] . \qquad [ Equation 3 ]$$

In Equation 3 above, λᵥₑₙₜ indicates an amount of infectious particles emitted by ventilation, λ_{RH} indicates an amount of natural pathogen inactivation by relative humidity, λ_{dep} indicates an amount of pathogens falling by gravity, λ_{PAC} indicates an amount of pathogens removed by an indoor quarantine air-conditioner, and λ_{rec} indicates an amount of pathogens removed by a building circulation air conditioner.

According to the above configuration, it is possible to accurately calculate the airborne infectious disease-infection risk which may be changed based on various variables in the quarantine target space. When using the Equations configured as above, it is possible to calculate the infection risk based on an air quality condition changed in real time depending on building, space, air conditioner, or the like, thus providing the method applicable to quarantine air-conditioning for preventing a spread of airborne infectious diseases even in an abnormal situation.
Step v) indicates step (S410) of instructing, by the quarantine air-conditioning central control unit 120, a quarantine air-conditioner 130 to perform an enhanced operation when the calculated airborne infectious disease-infection risk is the predetermined reference (threshold value) or higher (S400).

The method may include step (S420) of instructing, by the quarantine air-conditioning central control unit 120, the quarantine air-conditioner 130 to perform a normal operation when the calculated airborne infectious disease-infection risk is lower than the predetermined reference (threshold value) (S400).
Step vi) indicates step (S430) of notifying, by the quarantine air-conditioning central control unit 120, the airborne infectious disease-infection risk.

According to the above configuration, it is possible to accurately receive the air quality information detected at a site from a remote location, and thus continuously monitor the airborne infectious disease-infection risk at the remote location. Therefore, it is possible to predict the wide spread of the airborne infectious diseases in the remote location, and provide risk information, such as an evacuation instruction, to the site, thereby minimizing human damage caused by the wide spread airborne infectious diseases.

[System for Predicting Infection with Airborne Infectious Disease]

Components of this system that are the same as or similar to those of the quarantine air-conditioning system for preventing a spread of airborne infectious diseases described above are denoted by the same reference numerals throughout the drawings, and their redundant descriptions are omitted.

Referring to FIG. 1, the system for predicting infection with airborne infectious disease according to still another aspect of the present disclosure may include an air quality detection unit 110 detecting an air quality of a quarantine air-conditioning target space; and a quarantine air-conditioning central control unit 120 predicting an airborne infectious disease-infection risk based on air quality information detected by the air quality detection unit 110 and past air quality information already input.

The air quality detection unit 110 may include a complex environmental sensor 112 and a pathogen sensor 114.

The complex environmental sensor 112 of the air quality detection unit 110 may detect information to calculate the airborne infectious disease-infection risk, and transmit the detected information to the quarantine air-conditioning central control unit 120.

The present disclosure is not limited thereto, and the information detected by the complex environmental sensor 112 of the air quality detection unit 110 and then transmitted to the quarantine air-conditioning central control unit 120 may include all information helpful in predicting the airborne infectious disease-infection risk.

The present disclosure is not limited thereto, and the information may include information on a temperature, humidity, carbon dioxide, a total volatile organic compound (TVOC), a fine particle (PM), or an occupant.

The temperature, humidity, carbon dioxide, total volatile organic compound (TVOC), fine particle (PM), or occupant information may affect the survival and spread of the pathogen which is a cause of the airborne infectious disease, affect the infectious particle (Quanta) inhalation and respiration volume of the occupant, thereby affecting an infection probability.

The present disclosure is not limited thereto, and the quarantine air-conditioning central control unit may predict the airborne infectious disease-infection risk by an indoor air quality prediction algorithm based on time series analysis.

The present disclosure is not limited thereto, and the indoor air quality prediction algorithm may be based on autoregressive integrated moving average (ARIMA).

### Experimental Example

### Development of PM2.5 Concentration Prediction Model Using ARIMA Model

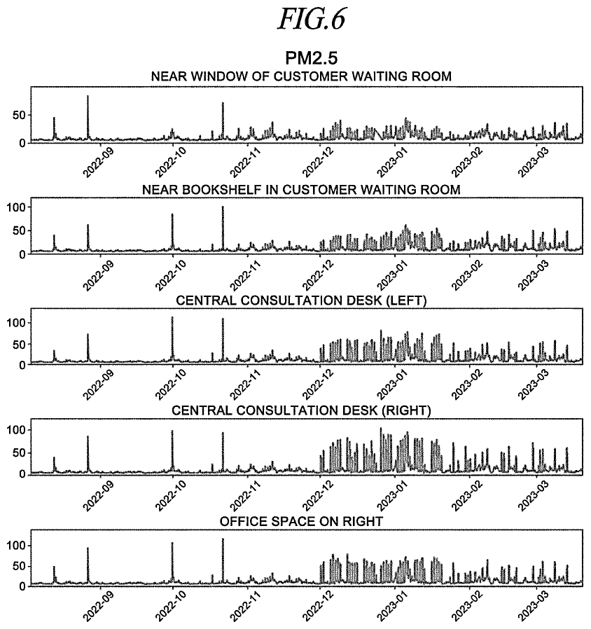

PM2.5 data was monitored by attaching five complex environmental sensors to the Gyeongsangnam-do Provincial Office from Aug. 3, 2022 to March 2023, and its results are shown in FIGS. 6 to 10.

That is, FIG. 6 is a graph showing the PM2.5 sensor data based on a time for each zone of the Gyeongsangnam-do Provincial Government Civil Affairs Office.

Referring to FIG. 6, the PM2.5 sensor data was analyzed by referring to data measured at five locations including two customer waiting rooms, two central consultation counters, and one office space on the right.

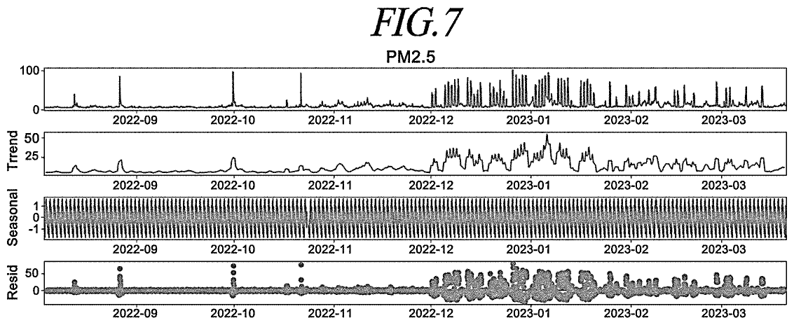

FIG. 7 is a graph showing time series decomposition in the PM2.5 sensor data of the Gyeongsangnam-do Provincial Government Civil Affairs Office.

Referring to FIG. 7, the trend, seasonal volatility, and irregular element (or residual) of the PM2.5 data may be checked as a result of conducting the time series decomposition on the PM2.5 sensor data expected to be highly related to the concentration of the airborne infectious pathogens.

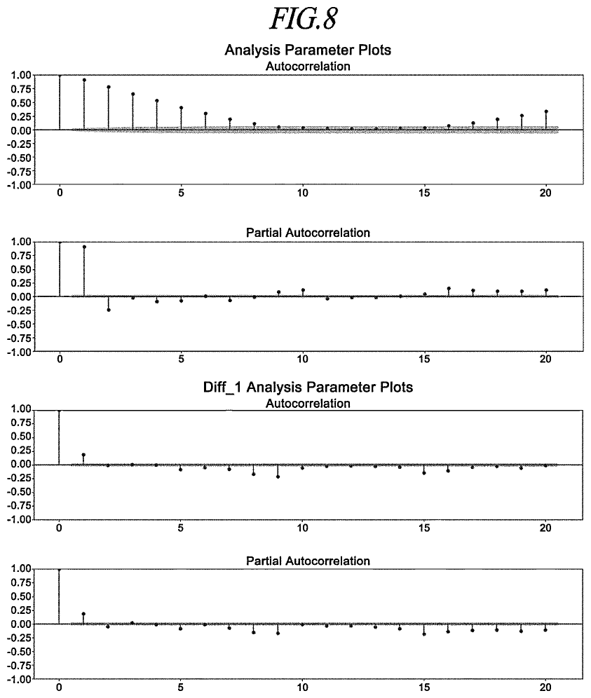

FIG. 8 is a graph showing time series stationarity (or auto correlation function (ACF)) in the PM2.5 sensor data of the Gyeongsangnam-do Provincial Government Civil Affairs Office.

Referring to FIG. 8, the time series stationarity (ACF) was confirmed from the PM2.5 sensor data, and it is thus seen that the PM2.5 sensor data is valid data that may be used for algorithm calculation.

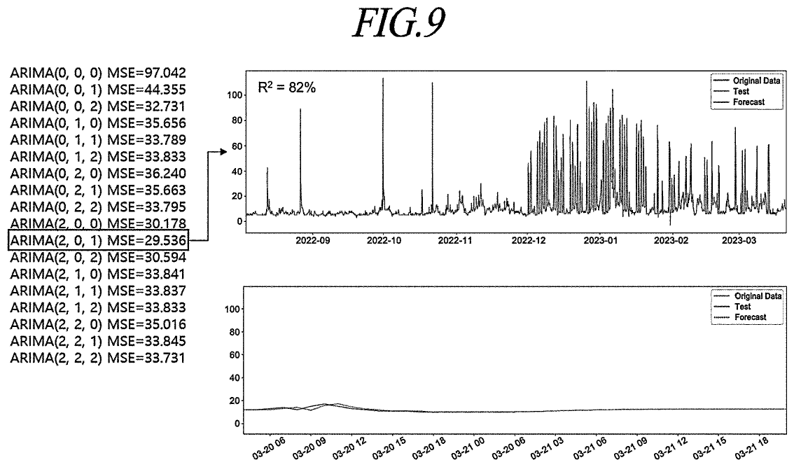

FIG. 9 is a graph showing a mean squared error of an ARIMA(p,d,q) model according to an embodiment of the present disclosure.

Referring to FIG. 9, it may be seen that an optimal ARIMA is acquired at Mean Squared Error 29.536 of ARIMA(2,0,1). Significant data was acquired at R²=82% from the above analysis, and it may thus be seen that it is possible to predict the infection index by using environmental data.

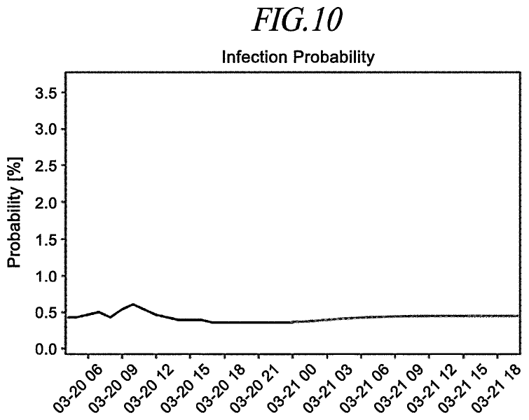

FIG. 10 is a graph showing the airborne infectious disease-infection probability and the airborne infectious disease-future infection probability based on various environments and parameters of a heating, ventilation, & air conditioning (HVAC) technology according to an embodiment of the present disclosure.

Referring to FIG. 10, it may be seen that data for predicting airborne SARS-COV-2 (B.1.1.7) infection probability based on a complex environment and the HVAC parameters is acquired under a condition of P (f (PM2.5), Q, Q_pac, 20%, 30 min). The real-time analysis and prediction of the infection probability shown in FIG. 10 is a result calculated based on a Quanta prediction empirical formula (f (PM2.5)) using the PM2.5 data, ventilation volume (Q), local air processing volume (Q_pac) of the quarantine air-conditioner, humidity of 20%, and residence time of 30 minutes.

The infection probability may be predicted from the PM2.5 data using the method as described above, which may use, as its parameter, not only the PM2.5 but also PM1.0, the temperature, the relative humidity, carbon dioxide, the TVOC, PM10, a pathogen type, the occupant information, or the like.

As set forth above, according to an embodiment of the present disclosure, the quarantine air-conditioning system for preventing a spread of airborne infectious diseases may include the complex environmental sensor and the pathogen sensor to thus accurately predict the airborne infectious disease-infection risk, thereby efficiently preventing the spread of the airborne infectious diseases.

According to an embodiment of the present disclosure, the quarantine air-conditioning system for preventing a spread of airborne infectious diseases may comprehensively reflect the feature of the pathogen which is the cause of the airborne infectious disease and interacts with the indoor environment, thereby more accurately predicting the infection risk of the infection with the airborne infectious disease.

According to an embodiment of the present disclosure, the quarantine air-conditioning method for preventing a spread of airborne infectious diseases may accurately predict the airborne infectious disease-infection risk to thus preemptively prevent the wide spread pathogens or notify the infection risk, thereby minimizing human casualties caused by the wide spread pathogens.

Hereinabove, specific portions of the present disclosure have been described in detail. However, it will be obvious to those skilled in the art that this detailed description is only a preferred embodiment and the scope of the present disclosure is not limited by this detailed description. Therefore, the substantial scope of the present disclosure will be defined by the accompanying claims and equivalents thereof.

What is claimed is:

1. A quarantine air-conditioning system for preventing a spread of airborne infectious diseases, the system comprising:
an air quality detection unit detecting an air quality of a quarantine air-conditioning target space;
a quarantine air-conditioning central control unit issuing a quarantine countermeasure instruction by calculating an airborne infectious disease-infection risk based on air quality information detected by the air quality detection unit; and
a quarantine air-conditioner operated based on the instruction issued by the quarantine air-conditioning central control unit,
wherein the air quality detection unit includes
a complex environmental sensor detecting information on a temperature, humidity, carbon dioxide, a total volatile organic compound (TVOC), particulate matter (PM), or an occupant, and
a pathogen sensor detecting airborne infectious pathogens.

2. The system of claim 1, wherein the pathogen sensor detects the bacteria, fungi, or viruses floating in indoor air, and transmits a detection result to the quarantine air-conditioning central control unit.

3. The system of claim 1, wherein the one or more air quality detection units are disposed in the quarantine air-conditioning target space, collect the air quality information at a predetermined time interval, and transmit the collected information to the quarantine air-conditioning central control unit.

4. The system of claim 1, wherein the quarantine air-conditioning central control unit includes an information processing unit calculating the infection risk for each airborne infectious disease based on the air quality information detected by the complex environmental sensor and the pathogen sensor.

5. The system of claim 1, wherein the airborne infectious disease-infection risk is based on an airborne infectious disease-infection probability calculated by Equation 1 below:
$$P = 1 - {e}^{- N} = 1 - {e}^{- IR \times n ( t ) \times t} , \qquad [ Equation 1 ]$$
where P indicates the infection probability (here, 1 indicates 100%), N indicates a total amount of infectious particles (Quanta) inhaled by one person, IR indicates an hourly gas respiration volume (m³/h) of an infected occupant, n(t) indicates Quanta concentration (m⁻³), and t indicates a residence time (h),
the Quanta concentration in Equation 1 is calculated by Equation 2 below:
$$n ( t ) = {n}_{0} {e}^{- \sum λ \times t} + \frac{S}{V \times \sum λ} \times ( 1 - {e}^{- \sum λ \times t} ) , \qquad [ Equation 2 ]$$
where n₀ indicates initial Quanta concentration, S indicates Quanta generation amount (1/h), V indicates a volume of an indoor space (m³), and Σλ indicates a total attenuation amount of pathogens per hour, and
the total attenuation amount of pathogens per hour in Equation 2 is calculated by Equation 3 below:
$$\sum λ = {λ}_{vent} + {λ}_{RH} + {λ}_{dep} + {λ}_{PAC} + {λ}_{rec} [ \frac{1}{h} ] , \qquad [ Equation 3 ]$$
where λᵥₑₙₜ indicates an amount of infectious particles emitted by ventilation, λ_{RH} indicates an amount of natural pathogen inactivation by relative humidity, λ_{dep} indicates an amount of pathogens falling by gravity, λ_{PAC} indicates an amount of pathogens removed by an indoor quarantine air-conditioner, and λ_{rec} indicates an amount of pathogens removed by a building circulation air conditioner.

6. The system of claim 1, wherein the airborne infectious disease-infection risk is based on a total attenuation amount of pathogens calculated by Equation 3 below:
$$\sum λ = {λ}_{vent} + {λ}_{RH} + {λ}_{dep} + {λ}_{PAC} + {λ}_{rec} [ \frac{1}{h} ] , \qquad [ Equation 3 ]$$
where λᵥₑₙₜ indicates an amount of infectious particles emitted by ventilation, λ_{RH} indicates an amount of natural pathogen inactivation by relative humidity, λ_{dep} indicates an amount of pathogens falling by gravity, λ_{PAC} indicates an amount of pathogens removed by an indoor quarantine air-conditioner, and λ_{rec} indicates an amount of pathogens removed by a building circulation air conditioner.

7. The system of claim 5, wherein the pathogens are one or more of viruses, bacteria, and fungi.

8. The system of claim 1, wherein the quarantine air-conditioning central control unit includes a receiver receiving the air quality information detected by the complex environmental sensor and pathogen sensor of the air quality detection unit.

9. The system of claim 1, wherein the quarantine air-conditioning central control unit includes a controller designed to instruct the quarantine air-conditioner to perform an enhanced quarantine air-conditioning operation when the airborne infectious disease-infection risk is a predetermined reference or higher.

10. The system of claim 1, wherein the quarantine air-conditioner receives the instruction from the quarantine air-conditioning central control unit to execute one or more of capture of the infectious particles, inactivation of the captured particles, dehumidification, and a temperature control.

11. The system of claim 1, wherein the quarantine air-conditioner is a fixed or mobile type, and includes the air quality detection unit.

12. The system of claim 1, wherein the quarantine air-conditioner is installed by analyzing the distribution and residence time of the particle spread under a building air-conditioning condition provided by air-conditioning equipment already installed in a building.

13. The system of claim 1, wherein the quarantine air-conditioning central control unit displays quarantine status notification in real time.

14. A quarantine air-conditioning method using a quarantine air-conditioning system for preventing a spread of airborne infectious diseases of claim 1, the method comprising:
i) operating a complex environmental sensor and a pathogen sensor;
ii) analyzing, by a quarantine air-conditioning central control unit, air quality information detected by the complex environmental sensor;
iii) analyzing, by the quarantine air-conditioning central control unit, a detection result of pathogens detected by the pathogen sensor;
iv) calculating, by the quarantine air-conditioning central control unit, an airborne infectious disease-infection risk based on analysis results of steps ii) and iii);
v) instructing, by the quarantine air-conditioning central control unit, a quarantine air-conditioner to perform an enhanced operation when the calculated airborne infectious disease-infection risk is a predetermined reference or higher; and
vi) notifying, by the quarantine air-conditioning central control unit, the airborne infectious disease-infection risk.

15. The method of claim 14, wherein the airborne infectious disease-infection risk is based on an airborne infectious disease-infection probability calculated by Equation 1 below:
$$P = 1 - {e}^{- N} = 1 - {e}^{- IR \times n ( t ) \times t} , \qquad [ Equation 1 ]$$
where P indicates the infection probability (here, 1 indicates 100%), N indicates a total amount of infectious particles (Quanta) inhaled by one person, IR indicates an hourly gas respiration volume (m³/h) of an infected occupant, n(t) indicates Quanta concentration (m⁻³), and t indicates a residence time (h),
the Quanta concentration in Equation 1 is calculated by Equation 2 below:
$$n ( t ) = {n}_{0} {e}^{- \sum λ \times t} + \frac{S}{V \times \sum λ} \times ( 1 - {e}^{- \sum λ \times t} ) , \qquad [ Equation 2 ]$$
where n₀ indicates initial Quanta concentration, S indicates Quanta generation amount (1/h), V indicates a volume of an indoor space (m³), and Σλ indicates a total attenuation amount of pathogens per hour, and
the total attenuation amount of pathogens per hour in Equation 2 is calculated by Equation 3 below:
$$\sum λ = {λ}_{vent} + {λ}_{RH} + {λ}_{dep} + {λ}_{PAC} + {λ}_{rec} [ \frac{1}{h} ] , \qquad [ Equation 3 ]$$
where λᵥₑₙₜ indicates an amount of infectious particles emitted by ventilation, λ_{RH} indicates an amount of natural pathogen inactivation by relative humidity, λ_{dep} indicates an amount of pathogens falling by gravity, λ_{PAC} indicates an amount of pathogens removed by an indoor quarantine air-conditioner, and λ_{rec} indicates an amount of pathogens removed by a building circulation air conditioner.

16. A system for predicting infection with airborne infectious diseases, the system comprising:
an air quality detection unit detecting an air quality of a quarantine air-conditioning target space; and
a quarantine air-conditioning central control unit predicting an airborne infectious disease-infection risk based on air quality information detected by the air quality detection unit and past air quality information already input,
wherein the air quality detection unit includes
a complex environmental sensor detecting information on a temperature, humidity, carbon dioxide, a total volatile organic compound (TVOC), particulate matter (PM), or an infected occupant, and
a pathogen sensor detecting airborne infectious pathogens, and
the quarantine air-conditioning central control unit predicts the airborne infectious disease-infection risk by an indoor air quality prediction algorithm based on time series analysis.

17. The system of claim 16, wherein the indoor air quality prediction algorithm is based on autoregressive integrated moving average (ARIMA).
